(19) United States
(12) Patent Application Publication
(10) Pub. No.: US 2025/0073765 A1
(43) Pub. Date: Mar. 6, 2025

(54) METHOD FOR REMEDYING POLYCYCLIC AROMATIC HYDROCARBONS (PAHS)-CONTAMINATED SOIL USING PSEUDOMONAS SP. KW-2

(71) Applicant: QINGDAO UNIVERSITY OF TECHNOLOGY, Qingdao (CN)

(72) Inventors: Huawei WANG, Qingdao (CN); Xiqing CUI, Qingdao (CN); Zhibo YANG, Qingdao (CN); Yanan WANG, Qingdao (CN); Xueqin WANG, Qingdao (CN); Rongxue ZOU, Qingdao (CN); Xiangyu MENG, Qingdao (CN); Yuewei YANG, Qingdao (CN); Yujun WANG, Qingdao (CN); Yingjie SUN, Qingdao (CN); Yuanwen LIU, Qingdao (CN); Haoran DING, Qingdao (CN)

(21) Appl. No.: 18/949,739

(22) Filed: Nov. 15, 2024

(30) Foreign Application Priority Data
Jan. 9, 2024 (CN) 202410029172.5

(51) Int. Cl. B09C 1/10; B09C 1/08; C12N 1/20; C12R 1/38

(52) U.S. Cl. CPC B09C 1/10; B09C 1/08; C12N 1/20; C12R 2001/38

(57) ABSTRACT

A microbial preparation and a method for remedying a polycyclic aromatic hydrocarbons (PAHs)-contaminated soil are disclosed. The microbial preparation contains Pseudomonas sp. KW-2 with a deposit number of CGMCC No. 29017. In the soil remediation method, an oxidizing agent is added to the PAHs-contaminated soil for pre-oxidation treatment, where the oxidizing agent is selected from the group consisting of Fenton's reagent, hydrogen peroxide, sodium persulfate, potassium permanganate, and a combination thereof; a carbon source or a nitrogen source added to reach a carbon-to-nitrogen ratio of 100:5-20, and the microbial preparation is introduced for microbial remediation, where the carbon source is selected from the group consisting of glucose, fructose, sucrose, starch, and a combination thereof; and the nitrogen source is selected from the group consisting of ammonium chloride, ammonium nitrate, potassium nitrate, sodium nitrate, and a combination thereof.

### CROSS-REFERENCE TO RELATED APPLICATIONS

This application claims the benefit of priority from Chinese Patent Application No. 202410029172.5, filed on Jan. 9, 2024. The content of the aforementioned application, including any intervening amendments thereto, is incorporated herein by reference in its entirety.

### TECHNICAL FIELD

This application relates to soil remediation, and more specifically to a method for remedying polycyclic aromatic hydrocarbons (PAHs)-contaminated soil using Pseudomonas sp. KW-2.

### BACKGROUND

The coking industry is closely related to industrial development. At the beginning of the 21^{st} century when the most rapid industrial growth occurred, a large number of coking plants were built, and the coke production experienced a dramatic growth. The coke production is accompanied by the generation of a variety of organic pollutants, which are dominated by polycyclic aromatic hydrocarbons (PAHs). PAHs are aromatic compounds containing two or more benzene rings, and can be categorized into three types according to the number of benzene rings, i.e., low-ring (2-3 ring) PAHs, medium-ring (4-ring) PAHs, and high-ring (5-6 ring) PAHs. PAHs intrinsically have high hydrophobicity and high melting and boiling points, and as the number of rings increases, the degradability becomes worse and worse, such that they are more likely to be deposited and gathered in the soil, which seriously affects the physical and chemical properties of the soil and threaten the soil ecosystem. PAHs can enter the human body through the food chain, and thus cause serious damages to the human body, as they have carcinogenic, teratogenic and mutagenic effects. The key pollutants prioritized for control by the U.S. Environmental Protection Agency include the following PAHs: low-ring (2-3 rings) PAHs including naphthalene (NaP), acenaphthylene (Ace), acenaphthene (Ac), fluorene (Fl), phenanthrene (Phe), anthracene (An); medium-ring (4-ring) PAHs including fluorescent anthracene (FLa), pyrene (Pyr), benzo(a)anthracene (BaA), hydrazine (Chr); and high-ring (5-6 rings) PAHs including benzo(b)fluoranthene (BbF), benzo (k) fluoranthene (BkF), benzo (a) pyrene (BaP), indeno (1,2,3-cd) pyrene (InD), dibenzo (a,h) anthracene (DahA), and benzo(g,h,i)perylene (BghiP). As PAHs have the characteristics of teratogenicity, carcinogenicity and mutagenicity, the contaminated soil surrounding the coking plants must be remedied for further exploitation.

Currently, remediation methods for PAHs-contaminated soil at coking sites mainly include leaching, chemical oxidation, and thermal desorption. Among them, the thermal desorption method has the advantages of high efficiency, short remediation cycle and wide applicability, but it has a high cost, and its remediation effect is susceptible to a variety of factors, such as temperature, pollutant content and soil type. Some studies have shown that clay particles are prone to agglomeration or thermal sclerosis, which makes the internal area difficult to be heated, thereby leading to a low remediation efficiency. For the leaching method, a leaching agent is adopted to dissolve the pollutants in the soil to achieve the pollutant removal, but it also has a high cost, and fails to effectively remove high-ring PAHs. Moreover, it may cause secondary pollution.

The chemical oxidation method uses an oxidizing agent to oxidize organic contaminants in soil into less toxic or non-toxic substances. Commonly used oxidizing agents include hydrogen peroxide, Fenton's reagent, permanganate, persulfate and ozone. It has been demonstrated that although the chemical oxidation method is more effective than other methods, in the actual application, the oxidization effect of different oxidizers on PAHs varies greatly, and some oxidizers can meet the remediation goals only at high doses, which may increase the treatment cost and lead to other problems.

The microbial remediation method has the advantages of low cost, convenient operation and controllable process. The existing microbial remediation technologies, such as bio-stimulation and bio-pile, generally have the defects of poor microbial adaptability, long remediation cycle and low efficiency. Therefore, the development of efficient, green, economic and safe remediation technologies of PAHs-contaminated soil has become a key issue to be solved for the coking sites.

### SUMMARY

An objective of the present disclosure is to provide a method for remedying polycyclic aromatic hydrocarbons (PAHs)-contaminated soil using a Pseudomonas sp. KW-2 to solve the above problems in the prior art.

The Pseudomonas sp. KW-2 strain has been deposited in China General Microbiological Culture Collection Center (CGMCC) (No. 1 West Beichen Road, Chaoyang District, Beijing 100101, China) on Nov. 15, 2023, with a deposit number of CGMCC No.29017.

The technical solutions of the present disclosure are as follows.

In a first aspect, this application provides a microbial preparation for remedying a polycyclic aromatic hydrocarbons (PAHs)-contaminated soil, wherein an active ingredient of the microbial preparation is Pseudomonas sp. KW-2 with a deposit number of CGMCC No.29017.

In a second aspect, this application provides a method for preparing the aforementioned microbial preparation, comprising:
inoculating a Pseudomonas sp. KW-2 strain into a nutrient broth medium for culture at 25-30° C. and 120-150 rpm for 1-3 days;
inoculating the nutrient broth medium into a peptone-yeast extract calcium maintenance (PYCM) medium for culture at 25-30° C. and 120-150 rpm for 1-3 days to obtain a bacterial solution; and
subjecting the bacterial solution to centrifugation, followed by removal of supernatant to obtain the microbial preparation.

In a third aspect, this application provides a method for remedying a polycyclic aromatic hydrocarbons (PAHs)-contaminated soil using the aforementioned microbial preparation, comprising:
adding an oxidizing agent to the PAHs-contaminated soil for pre-oxidation treatment to obtain a pre-oxidized soil; and
adding a carbon source or a nitrogen source to the pre-oxidized soil to reach a carbon-to-nitrogen ratio of 100:5-20, and introducing the aforementioned microbial preparation for microbial remediation;
wherein the oxidizing agent is selected from the group consisting of Fenton's reagent, hydrogen peroxide, sodium persulfate, potassium permanganate, and a combination thereof;
the carbon source is selected from the group consisting of glucose, fructose, sucrose, starch, and a combination thereof; and
the nitrogen source is selected from the group consisting of ammonium chloride, ammonium nitrate, potassium nitrate, sodium nitrate, and a combination thereof.

In this application, the term “carbon-to-nitrogen ratio” refers to the mass ratio of carbon to nitrogen contained in the soil, and the term “liquid-to-solid ratio” refers to the mass ratio of liquid to solid in the reaction system.

In an embodiment, the pre-oxidation treatment is performed through steps of:
adding a potassium permanganate solution to the PAHs-contaminated soil at a dose of 0.05-0.25 mmol/g; and adjusting a liquid-to-solid ratio of the PAHs-contaminated soil to 0.5-2:1 with water, followed by stirring and reaction.

In an embodiment, an additional amount of the microbial preparation is 50-100 mL per kg of the PAHs-contaminated soil.

In an embodiment, the microbial remediation is performed through steps of:
adding the microbial preparations to the pre-oxidized soil, followed by stirring and then reaction at 25-30° C. and a humidity of 40-50% for 3-21 days.

In an embodiment, the pre-oxidation treatment is performed at 20-35° C. and a humidity of 35-65% for 1-3 days.

The beneficial effects of the present disclosure are described as follows.

Pseudomonas sp. KW-2 is prepared into a microbial preparation, which is in combination with pre-oxidation treatment to realize the efficient remediation of PAHs-contaminated soil at coking sites, and the ecological function of the soil can be restored after remediation. The degradation mechanism of PAHs includes two stages: (1) during the pre-oxidation process, PAHs, especially the high-ring PAHs, will be partially degraded into non-PAHs substances by hydrogen peroxide and potassium permanganate, which is conducive to the subsequent biological treatment, and also can alleviate toxic effects on the microbial preparation; (2) during the microbial enhancement process, manganese oxidizing bacteria (i.e., the Pseudomonas sp. KW-2) and indigenous microorganisms can degrade PAHs through co-metabolism using PAHs as carbon and energy sources; and (3) during the microbial enhancement process, biogenic manganese oxides generated by the manganese oxidizing bacteria and transition state substances (such as dissolved Mn (III) ions, superoxide radicals, and hydrogen peroxide) generated during the growth and metabolism processes of the manganese oxidizing bacteria can also oxidize and degrade PAHs. As a result, the PAHs in the polluted soil surrounding the coking sites can be effectively degraded.

### DETAILED DESCRIPTION OF EMBODIMENTS

Terms used in the present disclosure, unless otherwise indicated, generally have the same meanings as commonly understood by those of ordinary skill in the art.

The present disclosure will be described in further detail below with reference to specific embodiments and data. The following embodiments are only exemplary, and are not intended to limit the scope of the present disclosure in any way.

EXAMPLE 1

Provided herein was a microbial preparation for treating a polycyclic aromatic hydrocarbons (PAHs)-contaminated soil at a coking site, which included a manganese-oxidizing bacterium as active ingredient.

The manganese-oxidizing bacterium was Pseudomonas sp. KW-2, which was isolated from a steel pipe rust sample from an experimental building of Qingdao University of Technology (Qingdao, Shandong, China), and was deposited in the China General Microbiological Culture Collection Center (CGMCC, No.3 building, No. 1 West Beichen Road, Chaoyang District, Beijing, China) on Nov. 15, 2023, with a deposit number of CGMCC No. 29017.

The above microbial preparation was prepared through the following steps.

A nutrient broth medium was prepared, into which the Pseudomonas sp. KW-2 was inoculated, cultured in a constant-temperature shaking incubator at 150 rpm and room temperature for 3 days, and transferred to a PYCM medium at an inoculation ratio of 2%. After that, the PYCM medium was cultured in the constant-temperature shaking incubator at 150 rpm and room temperature for 3 days to obtain a bacterial solution.

After the culture was completed, the bacterial solution was transferred to a centrifuge tube and centrifuged at 5000 r/min for 10 min, and the supernatant was discarded, and the precipitate was collected as the microbial preparation.

The nutrient broth medium was LB Broth Powder (Miller), which was purchased from Sangon Biotech (Shanghai) Co., Ltd.

The PYCM medium included 0.8 g/L of peptone, 0.2 g/L of yeast extract powder, 0.1 g/L of dipotassium hydrogen phosphate, 0.2 g/L of magnesium sulfate, 0.2 g/L of sodium nitrate, 0.1 g/L of calcium chloride, 0.1 g/L of ammonium chloride, 1.0 g/L of ferric ammonium citrate, and 0.1 g/L of ammonium carbonate.

EXAMPLE 2

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.05 mmol/g of a potassium permanganate solution (0.4 mol/L) was added. The system was adjusted to a liquid-to-solid ratio (weight ratio) of 1:1, uniformly stirred and transferred to an incubator for reaction at 25° C. and a humidity of 40% for 2 days.

(2) Adjustment of carbon-to-nitrogen ratio

The carbon-to-nitrogen ratio of the original soil was measured, where the organic carbon content was 15.73 g/kg, and the total nitrogen content was 4.21 g/kg, indicating that the soil was deficient in the organic carbon source and sufficient in the

N source, and the original organic carbon in the soil could not be utilized by the microorganisms. Hence, glucose was supplemented as the carbon source to reach a carbon-to-nitrogen ratio (weight ratio) of 100:10.

(3) Enhanced bio-remediation

The soil in step (2) was added with the microbial preparation prepared in Example 1 at a dose of 70 mL/kg (i.e., 70 mL of the microbial preparation was added per kilogram of the soil), stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment--Determination of polycyclic aromatic hydrocarbons-High performance liquid chromatography”, and the results were shown in Table 1.

TABLE 1
Removal rate of polycyclic aromatic hydrocarbons
from the soil through the treatment in Example 2

| | Content in | Content after the | Content after the | Removal |
|---|---|---|---|---|
| | the original | pre-oxidation | biological | rate |
| Contaminant | soil (mg/kg) | treatment (mg/kg) | treatment(mg/kg) | (%) |
| Benzo[a]anthracene | 9.01 | 4.52 | 2.04 | 77.36 |
| Benzo[a]pyrene | 9.60 | 4.62 | 1.82 | 81.04 |
| Benzo[1,2,3-cd] | 3.40 | 2.56 | 0.92 | 72.94 |
| pyrene | | | | |
| Total polycyclic | 108.3 | 55.56 | 19.34 | 82.14 |
| aromatic | | | | |
| hydrocarbons (PAHs) | | | | |

It could be seen from Table 1 that by using the microbial preparation of Example 1 for remedying PAHs in a coking contaminated soil, the content of benzo [a] anthracene in the soil was reduced from the initial 9.01 mg/kg to 2.04 mg/kg, with a degradation efficiency of 77.36%; the content of benzo [a] pyrene was reduced from the initial 9.60 mg/kg to 1.82 mg/kg, with a degradation efficiency of 81.04%; the content of benzo [1,2,3-cd] pyrene was reduced from the initial 3.40 mg/kg to 0.92 mg/kg, with a degradation efficiency of 72.94%; and the total PAHs was reduced from 108.3 mg/kg to 19.34 mg/kg with a degradation efficiency of 82.14%. Therefore, it could be concluded that the microbial preparation of Example 1 showed a good remediation effect on PAHs in soil.

EXAMPLE 3

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.08 mmol/g of a potassium permanganate solution (0.3 mol/L) was added. The system was adjusted to a liquid-to-solid ratio (weight ratio) of 2:1, uniformly stirred and transferred to an incubator for reaction at 25° C. and a humidity of 40% for 1 day.

(2) Adjustment of carbon-to-nitrogen ratio

The carbon-to-nitrogen ratio of the original soil was measured, where the organic carbon content was 15.73 g/kg, and the total nitrogen content was 4.21 g/kg, indicating that the soil was deficient in the organic carbon source and sufficient in the N source, and the original organic carbon in the soil could not be utilized by the microorganisms. Hence, glucose was supplemented as the carbon source to reach a carbon-to-nitrogen ratio (weight ratio) of 100:15.

(3) Enhanced bio-remediation

The soil in step (2) was added with the microbial preparation prepared in Example 1 at a dose of 70 mL/kg, stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment--Determination of polycyclic aromatic hydrocarbons-High performance liquid chromatography”, and the results were shown in Table 2.

TABLE 2
Removal rate of polycyclic aromatic hydrocarbons
in the soil through the treatment in Example 3

| | | | Content after the | |
|---|---|---|---|---|
| | Content in | Content after the | biological | Removal |
| | the original | pre-oxidation | treatment | rate |
| Contaminant | soil (mg/kg) | treatment (mg/kg) | (mg/kg) | (%) |
| Benzo[a]anthracene | 9.01 | 4.56 | 2.17 | 75.92 |
| Benzo[a]pyrene | 9.60 | 5.12 | 1.32 | 86.25 |
| Benzo[1,2,3-cd] | 3.40 | 1.45 | 0.95 | 72.06 |
| pyrene | | | | |
| Total polycyclic | 108.3 | 56.74 | 20.93 | 80.67 |
| aromatic | | | | |
| hydrocarbons (PAHs) | | | | |

It could be seen from Table 2 that by using the microbial preparation of Example 1 for remedying PAHs in a coking contaminated soil, the content of benzo [a] anthracene in the soil was reduced from the initial 9.01 mg/kg to 2.14 mg/kg, with a degradation efficiency of 75.92%; the content of benzo [a] pyrene was reduced from the initial 9.60 mg/kg to 1.32 mg/kg, with a degradation efficiency of 86.25%; the content of benzo [1,2,3-cd] pyrene was reduced from the initial 3.40 mg/kg to 0.95 mg/kg, with a degradation efficiency of 72.06%; and the total PAHs was reduced from 108.3 mg/kg to 20.93 mg/kg with a degradation efficiency of 80.67%. Therefore, it could be concluded that the microbial preparation of Example 1 showed a good remediation effect on PAHs in soil.

EXAMPLE 4

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.10 mmol/g of a potassium permanganate solution (0.35 mol/L) was added. The system was adjusted to a liquid-to-solid ratio (weight ratio) of 1:1, uniformly stirred and transferred to an incubator for reaction at 25° C. and a humidity of 40% for 1 day.

(2) Adjustment of carbon-to-nitrogen ratio

The carbon-to-nitrogen ratio of the original soil was measured, where the organic carbon content was 15.73 g/kg, and the total nitrogen content was 4.21 g/kg, indicating that the soil was deficient in the organic carbon source and sufficient in the N source, and the original organic carbon in the soil could not be utilized by the microorganisms. Hence, glucose was supplemented as the carbon source to render the reach a carbon-to-nitrogen ratio (weight ratio) of 100:20.

(3) Enhanced bio-remediation

The soil in step (2) was added with the microbial preparation prepared in Example 1 at a dose of 70 mL/kg, stirred for 5 min, and then transferred to an incubator at constant temperature and humidity (25° C. and 40% humidity) for reaction for 14 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 3.

TABLE 3
Removal rate of polycyclic aromatic hydrocarbons
in the soil through the treatment in Example 4

| | | | Content after the | |
|---|---|---|---|---|
| | Content in | Content after the | biological | Removal |
| | the original | pre-oxidation | treatment | rate |
| Contaminant | soil (mg/kg) | treatment (mg/kg) | (mg/kg) | (%) |
| Benzo[a]anthracene | 9.01 | 4.56 | 2.14 | 76.25 |
| Benzo[a]pyrene | 9.60 | 4.87 | 1.06 | 88.96 |
| Total polycyclic | 108.3 | 55.65 | 21.05 | 80.56 |
| aromatic | | | | |
| hydrocarbons (PAHs) | | | | |

It could be seen from Table 3 that by using the microbial preparation of Example 1 for remedying PAHs in a coking contaminated soil, the content of benzo [a] anthracene in the soil was reduced from the initial 9.01 mg/kg to 2.14 mg/kg, with a degradation efficiency of 76.25%; the content of benzo [a] pyrene was reduced from the initial 9.60 mg/kg to 1.06 mg/kg, with a degradation efficiency of 88.96%; and the total PAHs was reduced from 108.3 mg/kg to 21.05 mg/kg with a degradation efficiency of 80.56%. Therefore, it could be concluded that the microbial preparation of Example 1 showed a good remediation effect on PAHs in soil.

EXAMPLE 5

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.15 mmol/g of a potassium permanganate solution (0.5 mol/L) was added. The system was adjusted to a liquid-to-solid ratio (weight ratio) of 0.5:1, uniformly stirred and transferred to an incubator for reaction at 25° C. and a humidity of 40% for 2 days. (2) Adjustment of carbon-to-nitrogen ratio

The carbon-to-nitrogen ratio of the original soil was measured, where the organic carbon content was 15.73 g/kg, and the total nitrogen content was 4.21 g/kg, indicating that the soil was deficient in the organic carbon source and sufficient in the N source, and the original organic carbon in the soil could not be utilized by the microorganisms. Hence, glucose was supplemented as the carbon source to reach a carbon-to-nitrogen ratio (weight ratio) of 100:15.

(3) Enhanced bio-remediation

The soil in step (2) was added with the microbial preparation prepared in Example 1 at a dose of 70 mL/kg, stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 4.

TABLE 4
Removal rate of polycyclic aromatic hydrocarbons
in the soil through the treatment in Example 5

| | Content in | | | |
|---|---|---|---|---|
| | the | | Content after the | |
| | original | Content after the | biological | Removal |
| | soil | pre-oxidation | treatment | rate |
| Contaminant | (mg/kg) | treatment (mg/kg) | (mg/kg) | (%) |
| Benzo[a]anthracene | 9.01 | 4.56 | 0.73 | 91.90 |
| Benzo[b]fluoranthene | 14.62 | 7.54 | 3.01 | 79.41 |
| Benzo[a]pyrene | 9.60 | 4.59 | 0.19 | 98.02 |
| Dibenzo[a, | 1.25 | 0.85 | 0.13 | 89.60 |
| h]anthracene | | | | |
| Benzo[1,2,3-cd] pyrene | 3.40 | 1.87 | 0.20 | 94.12 |
| Total polycyclic | 108.3 | 54.86 | 17.86 | 83.51 |
| aromatic hydrocarbons | | | | |
| (PAHs) | | | | |

It could be seen from Table 4 that by using the microbial preparation of Example 1 for remedying PAHs in a coking contaminated soil, the content of benzo [a] anthracene in the soil was reduced from the initial 9.01 mg/kg to 0.73 mg/kg, with a degradation efficiency of 91.90%; the content of benzo [b] fluoranthene was reduced from the initial 14.62 mg/kg to 3.01 mg/kg, with a degradation efficiency of 79.41%; the content of benzo[a] pyrene was reduced from the initial 9.60 mg/kg to 0.19 mg/kg, with a degradation efficiency of 98.02%; the content of dibenzo[a, h] anthracene was reduced from the initial 1.25 mg/kg to 0.13 mg/kg, with a degradation efficiency of 89.60%; the content of benzo[1,2,3-cd] pyrene was reduced from the initial 3.40 mg/kg to 0.20 mg/kg, with a degradation efficiency of 94.12%; and the total PAHs was reduced from 108.3 mg/kg to 17.86 mg/kg with a degradation efficiency of 83.51%. Therefore, it could be concluded that the microbial preparation of Example 1 showed a good remediation effect on PAHs in soil.

EXAMPLE 6

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.25 mmol/g of a potassium permanganate solution (0.45 mol/L) was added. The system was adjusted to a liquid-to-solid ratio (weight ratio) of 1:1, uniformly stirred and transferred to an incubator for reaction at 25° C. and a humidity of 40% for 2 days.

(2) Adjustment of carbon-to-nitrogen ratio

The carbon-to-nitrogen ratio of the original soil was measured, where the organic carbon content was 15.73 g/kg, and the total nitrogen content was 4.21 g/kg, indicating that the soil was deficient in the organic carbon source and sufficient in the N source, and the original organic carbon in the soil could not be utilized by the microorganisms. Hence, glucose was weighed and supplemented as the carbon source to reach a carbon-to-nitrogen ratio (weight ratio) of 100:15.

(3) Enhanced bio-remediation

The soil in step (2) was added with the microbial preparation prepared in Example 1 at a dose of 70 mL/kg, stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment--Determination of polycyclic aromatic hydrocarbons-High performance liquid chromatography”, and the results were shown in Table 5.

TABLE 5
Removal rate of polycyclic aromatic hydrocarbons
in the soil through the treatment in Example 6

| | Content in | | | |
|---|---|---|---|---|
| | the | | Content after the | |
| | original | Content after the | biological | Removal |
| | soil | pre-oxidation | treatment | rate |
| Contaminant | (mg/kg) | treatment (mg/kg) | (mg/kg) | (%) |
| Benzo[a]anthracene | 9.01 | 4.56 | 0.70 | 92.23 |
| Benzo[b]fluoranthene | 14.62 | 7.46 | 2.64 | 81.94 |
| Benzo[a]pyrene | 9.60 | 5.15 | 0.17 | 98.23 |
| Dibenzo[a, | 1.25 | 0.56 | 0.17 | 86.40 |
| h]anthracene | | | | |
| Benzo[1,2,3-cd] pyrene | 3.40 | 1.47 | 0.16 | 95.29 |
| Total polycyclic | 108.3 | 56.14 | 17.90 | 83.47 |
| aromatic hydrocarbons | | | | |
| (PAHs) | | | | |

It could be seen from Table 5 that by using the microbial preparation of Example 1 for remedying PAHs in a coking contaminated soil, the content of benzo [a] anthracene in the soil was reduced from the initial 9.01 mg/kg to 0.70 mg/kg, with a degradation efficiency of 92.23%; the content of benzo [b] fluoranthene was reduced from the initial 14.62 mg/kg to 2.64 mg/kg, with a degradation efficiency of 81.94%; the content of benzo [a] pyrene was reduced from the initial 9.60 mg/kg to 0.17 mg/kg, with a degradation efficiency of 98.23%; the content of dibenzo [a, h] anthracene was reduced from the initial 1.25 mg/kg to 0.17 mg/kg, with a degradation efficiency of 86.40%; the content of benzo [1,2,3-cd] pyrene was reduced from the initial 3.40 mg/kg to 0.16 mg/kg, with a degradation efficiency of 95.29%; and the total PAHs was reduced from 108.3 mg/kg to 17.90 mg/kg with a degradation efficiency of 83.47%. Therefore, it could be concluded that the microbial preparation of Example 1 showed a good remediation effect on PAHs in soil.

EXAMPLE 7

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.05 mmol/g (by weight of the soil) of a potassium permanganate solution (0.30 mol/L) was added. The system was adjusted to a liquid-to-solid ratio (weight ratio) of 1:1, uniformly stirred and transferred to an incubator for reaction at 25° C. and a humidity of 40% for 2 days.

(2) Adjustment of carbon-to-nitrogen ratio

The carbon-to-nitrogen ratio of the original soil was measured, where the organic carbon content was 47.85 g/kg, and the Kjeldahl nitrogen content was 4.18 g/kg, indicating that the soil was deficient in the nitrogen source and sufficient in the organic carbon, and the original nitrogen in the soil could not be utilized by the microorganisms. Hence, potassium nitrate was weighed and supplemented as the nitrogen source for the soil to reach a carbon-to-nitrogen ratio (weight ratio) of 100:10.

(3) Enhanced bio-remediation

The soil in step (2) was added with the microbial preparation prepared in Example 1 at a dose of 60 mL/kg, stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 6.

TABLE 6
Removal rate of polycyclic aromatic hydrocarbons
in the soil through the treatment in Example 7

| | Content in | Content after the | Content after the | Removal |
|---|---|---|---|---|
| | the original | pre-oxidation | biological | rate |
| Contaminant | soil (mg/kg) | treatment (mg/kg) | treatment (mg/kg) | (%) |
| Benzo[a]anthracene | 29.49 | 16.20 | 7.65 | 74.06 |
| Benzo[a]pyrene | 27.11 | 6.57 | 5.93 | 78.13 |
| Total polycyclic | 300.15 | 154.88 | 58.80 | 80.41 |
| aromatic | | | | |
| hydrocarbons (PAHs) | | | | |

It could be seen from Table 6 that the content of benzo [a] pyrene was reduced from the initial 27.11 mg/kg to 5.93 mg/kg, with a degradation efficiency of 78.13%; the content of benzo[a] anthracene in the soil was reduced from the initial 29.49 mg/kg to 7.65 mg/kg, with a degradation efficiency of 74.06%; and the total PAHs was reduced from 300.15 mg/kg to 58.80 mg/kg with a degradation efficiency of 80.41%. Therefore, it could be concluded that the microbial preparation of Example 1 showed a good remediation effect on PAHs in soil.

EXAMPLE 8

Provided herein was a method for remedying polycyclic aromatic hydrocarbons (PAHs) in a coking contaminated soil by using a microbial preparation containing Pseudomonas sp. KW-2, which included the following steps. (1) Pre-oxidation treatment

200.00 g of the coking contaminated soil was placed in a beaker, to which 0.08 mmol/g (by weight of the soil) of a potassium permanganate solution (0.40 mol/L) was added. The system was adjusted to a liquid-to-solid ratio (weight ratio) of 2:1, uniformly stirred and transferred to an incubator for reaction at 25° C. and a humidity of 40% for 1 day.

(2) Adjustment of carbon-to-nitrogen ratio

The carbon-to-nitrogen ratio of the original soil was measured, where the organic carbon content was 47.85 g/kg, and the Kjeldahl nitrogen content was 4.18 g/kg, indicating that the soil was deficient in the nitrogen source and sufficient in the organic carbon, and the original nitrogen in the soil could not be utilized by the microorganisms. Hence, potassium nitrate was weighed and supplemented as the nitrogen source for the soil to reach a carbon-to-nitrogen ratio (weight ratio) of 100:15.

(3) Enhanced bio-remediation

The soil in step (2) was added with the microbial preparation prepared in Example 1 at a dose of 60 mL/kg, stirred for 5 min, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 7.

TABLE 7
Removal rate of polycyclic aromatic hydrocarbons
in the soil through the treatment in Example 8

| | | | Content after the | |
|---|---|---|---|---|
| | Content in | Content after the | biological | Removal |
| | the original | pre-oxidation | treatment | rate |
| Contaminant | soil (mg/kg) | treatment (mg/kg) | (mg/kg) | (%) |
| Benzo[a]anthracene | 29.49 | 11.61 | 6.15 | 79.15 |
| Benzo[a]pyrene | 27.11 | 3.96 | 3.73 | 86.24 |
| Benzo[1,2,3-cd] | 18.46 | 9.09 | 4.43 | 76.00 |
| pyrene | | | | |
| Total polycyclic | 300.15 | 126.96 | 58.76 | 80.42 |
| aromatic | | | | |
| hydrocarbons (PAHs) | | | | |

It could be seen from Table 7 that the content of benzo [a] pyrene was reduced from the initial 29.49 mg/kg to 6.15 mg/kg, with a degradation efficiency of 79.15%; the content of benzo [a] anthracene in the soil was reduced from the initial 27.11 mg/kg to 3.73 mg/kg, with a degradation efficiency of 86.24%; the content of benzo[1,2,3-cd] pyrene in the soil was reduced from the initial 18.46 mg/kg to 4.43 mg/kg, with a degradation efficiency of 76.00%; and the total PAHs was reduced from 300.15 mg/kg to 58.76 mg/kg with a degradation efficiency of 80.42%. Therefore, it could be concluded that the microbial preparation of Example 1 showed a good remediation effect on PAHs in soil.

EXAMPLE 9

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.10 mmol/g (by weight of the soil) of a potassium permanganate solution (0.35 mol/L) was added. The system was adjusted to a liquid-to-solid ratio (weight ratio) of 1:1, uniformly stirred and transferred to an incubator for reaction at 25° C. and a humidity of 40% for 1 day.

(2) Adjustment of carbon-to-nitrogen ratio

The carbon-to-nitrogen ratio of the original soil was measured, where the organic carbon content was 47.85 g/kg, and the Kjeldahl nitrogen content was 4.18 g/kg, indicating that the soil was deficient in the nitrogen source and sufficient in the organic carbon, and the original nitrogen in the soil could not be utilized by the microorganisms. Hence, potassium nitrate was supplemented as the nitrogen source to reach a carbon-to-nitrogen ratio (weight ratio) of 100:20.

(3) Enhanced bio-remediation

The soil in step (2) was added with the microbial preparation prepared in Example 1 at a dose of 60 mL/kg, stirred uniformly, and then transferred to an incubator at constant temperature and humidity (25° C. and 40% humidity) for reaction for 14 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 8.

TABLE 8
Removal rate of polycyclic aromatic hydrocarbons
in the soil through the treatment in Example 9

| | Content in | Content after the | Content after the | Removal |
|---|---|---|---|---|
| | the original | pre-oxidation | biological | rate |
| Contaminant | soil (mg/kg) | treatment (mg/kg) | treatment (mg/kg) | (%) |
| Benzo[a]anthracene | 29.49 | 8.00 | 6.26 | 78.77 |
| Benzo[a]pyrene | 27.11 | 4.25 | 2.40 | 91.15 |
| Benzo[1,2,3-cd] | 18.46 | 6.57 | 4.13 | 77.63 |
| pyrene | | | | |
| Total polycyclic | 300.15 | 104.81 | 58.75 | 80.43 |
| aromatic | | | | |
| hydrocarbons (PAHs) | | | | |

It could be seen from Table 8 that the content of benzo [a] pyrene was reduced from the initial 29.49 mg/kg to 6.26 mg/kg, with a degradation efficiency of 78.77%; the content of benzo [a] anthracene in the soil was reduced from the initial 27.11 mg/kg to 2.40 mg/kg, with a degradation efficiency of 91.15%; the content of benzo [1,2,3-cd] pyrene in the soil was reduced from the initial 18.46 mg/kg to 4.13 mg/kg, with a degradation efficiency of 77.63%; and the total PAHs was reduced from 300.15 mg/kg to 58.75 mg/kg with a degradation efficiency of 80.43%. Therefore, it could be concluded that the microbial preparation of Example 1 showed a good remediation effect on PAHs in soil.

EXAMPLE 10

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.15 mmol/g (by weight of the soil) of a potassium permanganate solution (0.40 mol/L) was added. The system was adjusted to a liquid-to-solid ratio (weight ratio) of 0.5:1, uniformly stirred and transferred to an incubator for reaction at 25° C. and a humidity of 40% for 2 days.

(2) Adjustment of carbon-to-nitrogen ratio

The carbon-to-nitrogen ratio of the original soil was measured, where the organic carbon content was 47.85 g/kg, and the Kjeldahl nitrogen content was 4.18 g/kg, indicating that the soil was deficient in the nitrogen source and sufficient in the organic carbon, and the original nitrogen in the soil could not be utilized by the microorganisms. Hence, potassium nitrate was supplemented as the nitrogen source to reach a carbon-to-nitrogen ratio (weight ratio) of 100:15.

(3) Enhanced bio-remediation

The soil in step (2) was added with the microbial preparation prepared in Example 1 at a dose of 60 mL/kg, stirred uniformly, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment--Determination of polycyclic aromatic hydrocarbons-High performance liquid chromatography”, and the results were shown in Table 9.

TABLE 9
Removal rate of polycyclic aromatic hydrocarbons
in the soil through the treatment in Example 10

| | Content in | | | |
|---|---|---|---|---|
| | the | | Content after the | |
| | original | Content after the | biological | Removal |
| | soil | pre-oxidation | treatment | rate |
| Contaminant | (mg/kg) | treatment (mg/kg) | (mg/kg) | (%) |
| Benzo[a]anthracene | 29.49 | 3.24 | 3.17 | 89.25 |
| Benzo[b]fluoranthene | 43.44 | 9.42 | 9.38 | 78.41 |
| Benzo[a]pyrene | 27.11 | 0.54 | 0.53 | 98.05 |
| Dibenzo[a, | 5.45 | 0.86 | 0.79 | 85.50 |
| h]anthracene | | | | |
| Benzo[1,2,3-cd] pyrene | 18.46 | 1.34 | 1.23 | 93.34 |
| Total polycyclic | 300.15 | 53.99 | 50.04 | 83.33 |
| aromatic hydrocarbons | | | | |
| (PAHs) | | | | |

It could be seen from Table 9 that the content of benzo [a] anthracene was reduced from the initial 29.49 mg/kg to 3.17 mg/kg, with a degradation efficiency of 89.25%; the content of benzo [b] fluoranthene was reduced from the initial 43.44 mg/kg to 9.38 mg/kg, with a degradation efficiency of 78.41%; the content of benzo [a] pyrene in the soil was reduced from the initial 27.11 mg/kg to 0.53 mg/kg, with a degradation efficiency of 98.05%; the content of dibenzo [a, h] anthracene in the soil was reduced from the initial 5.45 mg/kg to 0.79 mg/kg, with a degradation efficiency of 85.50%; the content of benzo [1,2,3-cd] pyrene in the soil was reduced from the initial 18.46 mg/kg to 1.23 mg/kg, with a degradation efficiency of 93.34%; and the total PAHs was reduced from 300.15 mg/kg to 50.04 mg/kg with a degradation efficiency of 83.33%. Therefore, it could be concluded that the microbial preparation of Example 1 showed a good remediation effect on PAHs in soil.

EXAMPLE 11

Provided herein was a method for remedying polycyclic aromatic hydrocarbons (PAHs) in a coking contaminated soil by using a microbial preparation containing Pseudomonas sp. KW-2, which included the following steps.

(1) Pre-oxidation treatment

400.00 g of the coking contaminated soil was placed in a beaker, to which 0.25 mmol/g (by weight of the soil) of a potassium permanganate solution (0.45 mol/L) was added. The system was adjusted to a liquid-to-solid ratio (weight ratio) of 1:1, uniformly stirred and transferred to an incubator for reaction at 25° C. and a humidity of 40% for 2 days.

(2) Adjustment of carbon-to-nitrogen ratio

The carbon-to-nitrogen ratio of the original soil was measured, where the organic carbon content was 47.85 g/kg, and the Kjeldahl nitrogen content was 4.18 g/kg, indicating that the soil was deficient in the nitrogen source and sufficient in the organic carbon, and the original nitrogen in the soil could not be utilized by the microorganisms. Hence, potassium nitrate was supplemented as the nitrogen source to reach a carbon-to-nitrogen ratio (weight ratio) of 100:10.

(3) Enhanced bio-remediation

The soil in step (2) was added with the microbial preparation prepared in

Example 1 at a dose of 60 mL/kg, stirred uniformly, and then transferred to an incubator at constant temperature and humidity (25° C. and 40% humidity) for reaction for 7 days.

The removal rate of polycyclic aromatic hydrocarbons was determined according to National Environmental Protection Standard HJ 784-2016 “Soil and sediment—Determination of polycyclic aromatic hydrocarbons-High performance liquid chromatography”, and the results were shown in Table 10.

TABLE 10
Removal rate of polycyclic aromatic hydrocarbons
in the soil through the treatment in Example 11

| | Content in | | | |
|---|---|---|---|---|
| | the | | Content after the | |
| | original | Content after the | biological | Removal |
| | soil | pre-oxidation | treatment | rate |
| Contaminant | (mg/kg) | treatment (mg/kg) | (mg/kg) | (%) |
| Benzo[a]anthracene | 29.49 | 3.10 | 2.93 | 90.06 |
| Benzo[b]fluoranthene | 43.44 | 8.52 | 8.47 | 80.50 |
| Benzo[a]pyrene | 27.11 | 0.56 | 0.55 | 97.97 |
| Dibenzo[a, | 5.45 | 0.82 | 0.80 | 85.32 |
| h]anthracene | | | | |
| Benzo[1,2,3-cd] pyrene | 18.46 | 0.95 | 0.93 | 94.96 |
| Total polycyclic | 300.15 | 52.28 | 50.18 | 83.28 |
| aromatic hydrocarbons | | | | |
| (PAHs) | | | | |

It could be seen from Table 10 that the content of benzo [a] anthracene was reduced from the initial 29.49 mg/kg to 2.93 mg/kg, with a degradation efficiency of 90.06%; the content of benzo [b] fluoranthene was reduced from the initial 43.44 mg/kg to 8.47 mg/kg, with a degradation efficiency of 80.50%; the content of benzo [a] pyrene in the soil was reduced from the initial 27.11 mg/kg to 0.55 mg/kg, with a degradation efficiency of 97.97%; the content of dibenzo[a, h] anthracene in the soil was reduced from the initial 5.45 mg/kg to 0.80 mg/kg, with a degradation efficiency of 85.32%; the content of benzo[1,2,3-cd] pyrene in the soil was reduced from the initial 18.46 mg/kg to 0.93 mg/kg, with a degradation efficiency of 94.96%; and the total PAHs was reduced from 300.15 mg/kg to 50.18 mg/kg with a degradation efficiency of 83.28%. Therefore, it could be concluded that the microbial preparation of Example 1 showed a good remediation effect on PAHs in soil.

In summary, the method using the microbial preparation provided in the present disclosure had a good remediation effect on the PAHs in the coking contaminated soil. Through the combination of pre-oxidative degradation, metabolic degradation by manganese-oxidizing bacteria and oxidative degradation by the manganese-oxidizing bacteria, the PAHs can be effectively removed from the coking contaminated soil.

It should be noted that the above embodiments are only preferred embodiments, and are not intended to limit the present disclosure in any other form. Modifications made by one of ordinary skill in the art by using the technical solutions of the present disclosure are considered as equivalent replacements. Any modifications, equivalent replacements, and improvements made within the spirit and principles of the present disclosure shall be included in the scope of the present disclosure defined by the appended claims.

What is claimed is:

1. A microbial preparation for remedying a polycyclic aromatic hydrocarbons (PAHs)-contaminated soil, wherein an active ingredient of the microbial preparation is Pseudomonas sp. KW-2 with a deposit number of CGMCC No. 29017.

2. A method for preparing the microbial preparation of claim 1, comprising:
inoculating a Pseudomonas sp. KW-2 strain into a nutrient broth medium for culture at 25-30° C. and 120-150 rpm for 1-3 days;
inoculating the nutrient broth medium into a peptone-yeast extract calcium maintenance (PYCM) medium for culture at 25-30° C. and 120-150 rpm for 1-3 days to obtain a bacterial solution; and
subjecting the bacterial solution to centrifugation, followed by removal of supernatant to obtain the microbial preparation.

3. A method for remedying a polycyclic aromatic hydrocarbons (PAHs)-contaminated soil, comprising:
adding an oxidizing agent to the PAHs-contaminated soil for pre-oxidation treatment to obtain a pre-oxidized soil; and
adding a carbon source or a nitrogen source to the pre-oxidized soil to reach a carbon-to-nitrogen ratio of 100:5-20, and introducing the microbial preparation of claim 1 for microbial remediation;
wherein the oxidizing agent is selected from the group consisting of Fenton's reagent, hydrogen peroxide, sodium persulfate, potassium permanganate, and a combination thereof;
the carbon source is selected from the group consisting of glucose, fructose, sucrose, starch, and a combination thereof; and
the nitrogen source is selected from the group consisting of ammonium chloride, ammonium nitrate, potassium nitrate, sodium nitrate, and a combination thereof.

4. The method of claim 3, wherein the pre-oxidation treatment is performed through steps of:
adding a potassium permanganate solution to the PAHs-contaminated soil at a dose of 0.05-0.25 mmol/g; and adjusting a liquid-to-solid ratio of the PAHs-contaminated soil to 0.5-2:1 with water, followed by stirring and reaction.

5. The method of claim 3, wherein an addition amount of the microbial preparation is 50-100 mL per kg of the PAHs-contaminated soil.

6. The method of claim 5, wherein the microbial remediation is performed through steps of:
adding the microbial preparation to the pre-oxidized soil, followed by stirring and reaction at 25-30° C. and a humidity of 40-50% for 3-21 days.

7. The method of claim 3, wherein the pre-oxidation treatment is performed at 20-35° C. and a humidity of 35-65% for 1-3 days.
